(19) United States
(12) Patent Application Publication
(10) Pub. No.: US 2025/0242101 A1
(43) Pub. Date: Jul. 31, 2025

(54) AUTOMATIC AT-HOME ACNE TREATMENT DEVICE

(71) Applicant: L'Oreal, Paris (FR)

(72) Inventor: Gracemarie Papaleo, Helmetta, NJ (US)

(73) Assignee: L'Oreal, Paris (FR)

(21) Appl. No.: 18/425,518

(22) Filed: Jan. 29, 2024

(51) Int. Cl. A61M 1/00; A61H 7/00; A61M 35/00; A61M 37/00

(52) U.S. Cl. CPC A61M 1/84; A61H 7/003; A61M 1/80; A61M 35/003; A61H 2201/105; A61M 2037/003; A61M 2205/051; A61M 2205/50; A61M 2205/6009; A61M 2210/04

(57) ABSTRACT

An applicator for treating acne including one or more microneedles, an optical sensor configured to detect acne, a removal system including a suction mechanism configured to remove acne material, and a chamber for containing the acne material, a remedy system configured apply one or more acne remedies, a light source configured to apply a light therapy to skin, and a processor configured to detect acne at one or more affected areas on skin, and direct the applicator to apply treatment, where the treatment includes penetrating the skin at the one or more affected areas with the one or more microneedles, removing acne material with suction mechanism, applying the light therapy to the one or more affected areas, and applying one or more acne remedies to the one or more affected areas.

### SUMMARY

Disclosed herein are systems, devices, and methods including an applicator for treating acne having one or more microneedles, an optical sensor configured to detect acne, a removal system including a suction mechanism configured to remove acne material, and a chamber for containing the acne material, a remedy system configured apply one or more acne remedies, a light source configured to apply a light therapy to skin, and a processor configured to detect acne at one or more affected areas on skin, and direct the applicator to apply treatment, where the treatment includes penetrating the skin at the one or more affected areas with the one or more microneedles, removing acne material with the suction mechanism, applying the light therapy to the one or more affected areas, and applying one or more acne remedies to the one or more affected areas.

In some embodiments, the applicator further includes a treatment plane configured to contact the skin, where the treatment plane surrounds the one or more microneedles. In some embodiments, the applicator further includes one or more massagers located on the treatment plane, wherein the one or more massagers are configured to massage the skin. In some embodiments, the treatment plane is rounded. In some embodiments, the light source is disposed on the treatment plane.

In some embodiments, the suction mechanism comprises a piezoelectric pump.

In some embodiments, the remedy system includes one or more reservoirs configured to hold the one or more remedies, and a valve configured to regulate the application of the one or more remedies to skin. In some embodiments, the valve is disposed on the treatment plane.

In some embodiments, the one or more microneedles are a plurality of microneedles arranged in an array.

In some embodiments, the light source is configured to emit blue light, near infrared (NIR) light, infrared (IR) light, ultraviolet (UV) light, or a combination thereof. In some embodiments, the light source is configured to emit an electromagnetic stimulus having a peak emission wavelength in the blue, near infrared (NIR), infrared (IR), ultraviolet (UV) spectrum, or a combination thereof.

Also disclosed herein is a system for treating acne, including an applicator as described herein, and a smart device communicatively coupled to the applicator.

In some embodiments, the smart device is configured to store treatment data of the applicator, wherein the treatment data comprises an instance of treatment. In some embodiments, the smart device is configured to anonymize, de-identify, or the like treatment data collected or stored therein. In some embodiments, the smart device is further configured to detect one or more indicators of irritation on the skin, and when the one or more indicators of irritation are detected, prevent treatment. In some embodiments, the smart device is further configured to determine an amount of time between a first treatment and a second treatment, compare the amount of time to a time threshold, and when the amount of time is below the time threshold, prevent treatment.

Also disclosed herein is a method of treating acne with the systems and devices as described herein, the method including detecting acne at one or more affected areas, applying treatment, wherein treatment includes penetrating skin with one or more microneedles, suctioning acne material from the acne, illuminating the one or more affected areas with light treatment, and applying one or more acne remedies to the one or more affected areas.

In some embodiments, the method further includes massaging the one or more affected areas.

In some embodiments, the method further includes storing treatment data, wherein the treatment data comprises an instance of the treatment.

In some embodiments, the method further includes detecting one or more indicators of irritation on the skin, and, when the one or more indicators of irritation are detected, preventing treatment.

In some embodiments, the method further includes determining an amount of time between a first treatment and a second treatment, comparing the amount of time to a time threshold, and, when the amount of time is below the time threshold, preventing treatment.

In some embodiments, the method further includes determining if a user of the applicator is a same user associated with the stored treatment, and, when the user is not the same user, preventing treatment.

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1A is an example applicator for treating acne, in accordance with the present technology;

FIG. 1B is a cross section of the example applicator of FIG. 1A, in accordance with the present technology;

FIG. 2 is a close-up cross-sectional view of an example applicator, in accordance with the present technology;

FIGS. 3A-3D are top-down perspectives of example applicator treatment planes, in accordance with the present technology;

FIGS. 4A-4B are side-view perspectives of example applicator treatment planes, in accordance with the present technology;

FIG. 5 is an example system for treating acne, in accordance with the present technology;

FIGS. 6A-6G are process diagrams of the applicator of FIG. 2 in use, in accordance with the present technology;

FIG. 7 is an example method of using the applicator and/or system described herein, in accordance with the present technology;

FIG. 8 is an example method of using the applicator and/or system described herein, in accordance with the present technology;

FIG. 9 is an example treatment method, in accordance with the present technology;

FIG. 10 is another example treatment method, in accordance with the present technology; and

FIG. 11 is yet another example treatment method, in accordance with the present technology.

### DETAILED DESCRIPTION

Disclosed herein are applicators, systems, and methods for treating acne at home. Deeply embedded acne or clogged pores can be hard to remove and clean without long term healing period or professional help. They can also be very unpleasant socially, painful, and cause swelling and inflammation. Below skin surface area (i.e., the epidermis) they become more difficult to remove and treat effectively. Described herein is a handheld applicator configured to precisely penetrate the skin with one or more microneedle directly over the affected area, and then suction out the bacteria (or acne material). In some embodiments, the applicator is further configured to emit a light stimulus (such as blue light) and dispense one or more remedies (or “formulas”) to kill additional bacterium, clean or sterilize an area, and promote further healing. In some embodiments, the one or more remedies may also prevent or reduce infection. In this manner, the devices and systems described herein reduce harmful practices such as constant attention, picking at existing acne, causing worse conditions or increased acne, inflammation, redness, scarring, darkness/hyperpigmentation, and spots/marks.

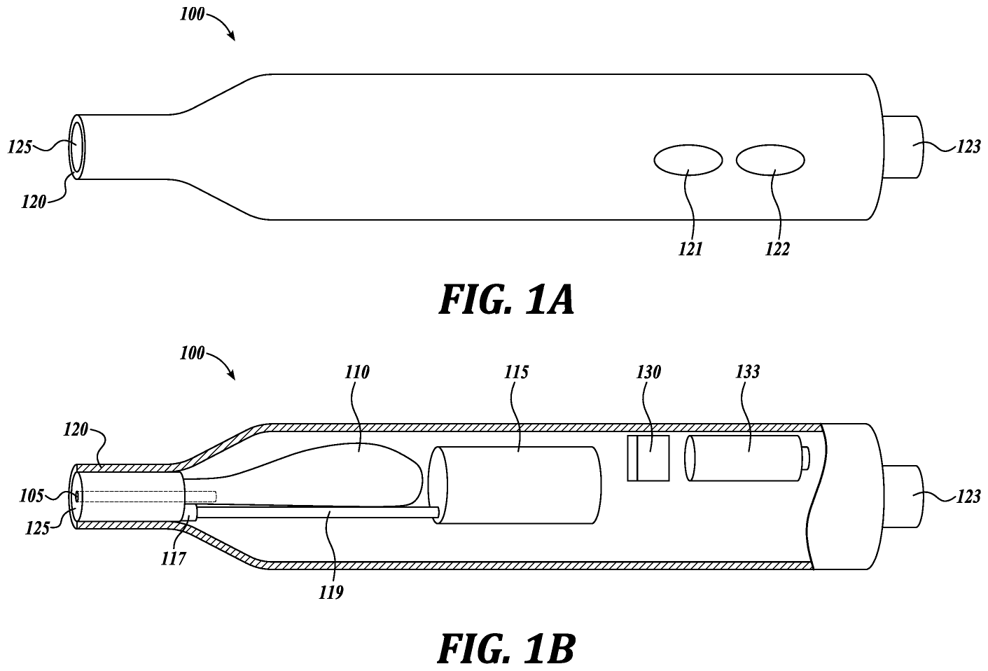

FIG. 1A is an example applicator 100 for treating acne, and the like in accordance with the present technology. In some embodiments, the applicator 100 includes a treatment plane 120 that surrounds an opening 125, a first actuator 121, a second actuator 122, and a button.

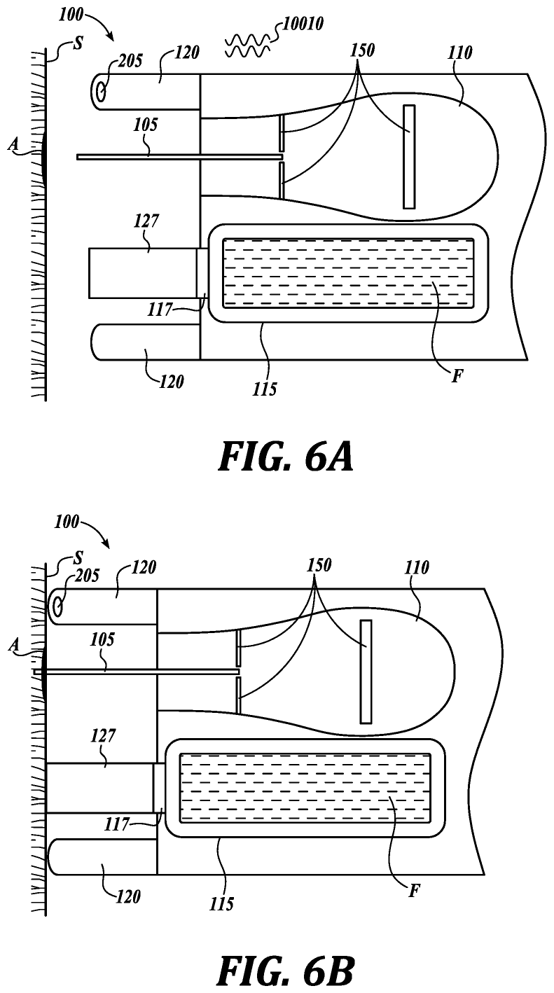

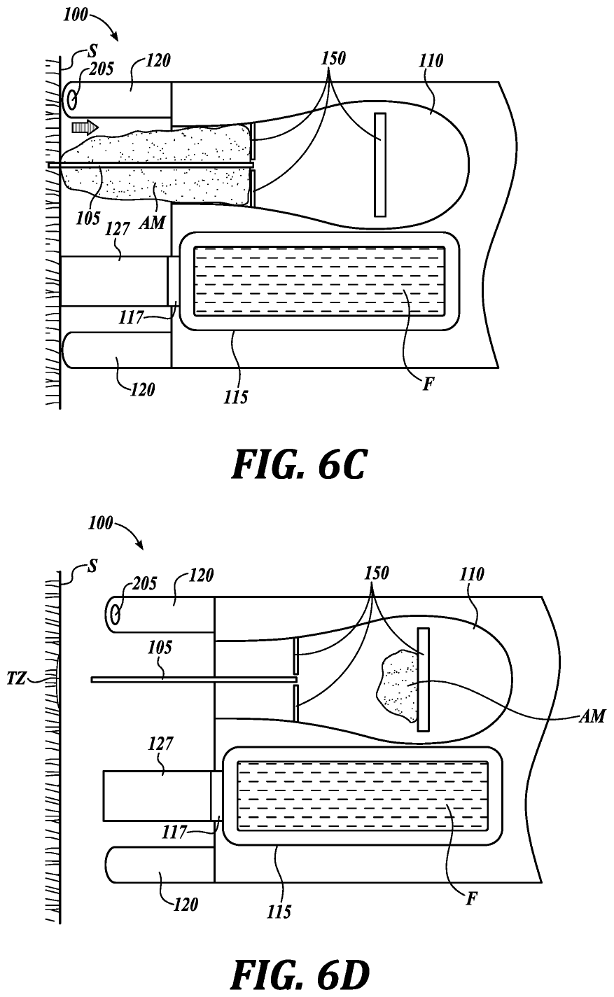

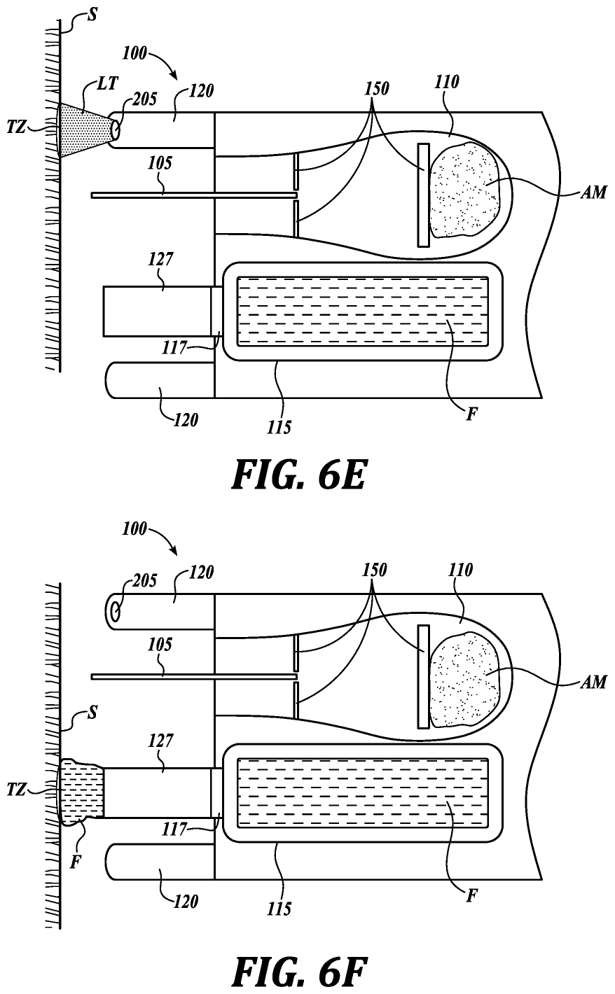

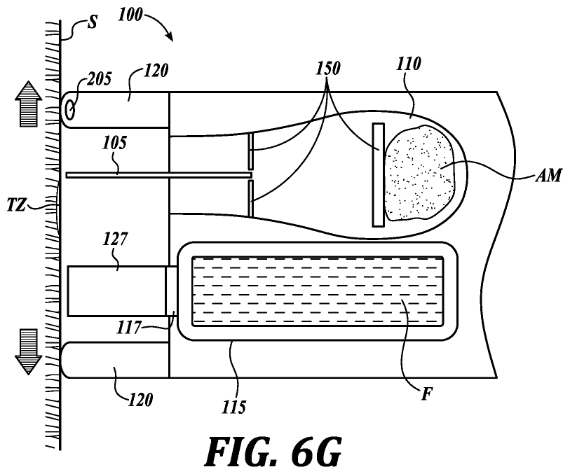

In some embodiments, the treatment plane 120 is configured to contact skin. In some embodiments, the treatment plane may be placed or pressed into skin, as shown in FIGS. 6A-6G. The treatment plane 120 may be configured to facilitate the application of treatment. As used herein, treatment includes penetrating the skin with one or more microneedles, suctioning acne material with a suction mechanism, applying light treatment to one or more affected areas, and/or applying one or more remedies (or “formulas”) to the affected areas. In some embodiments, treatment further includes massaging the skin. Examples of treatment are shown and described in detail in FIGS. 6A-6G, and FIGS. 7-11.

In some embodiments, one or more microneedle (as shown in FIG. 1B) is disposed inside the opening 125. In some embodiments, the opening 125 further allows for treatment to be applied to skin. For example, as shown in FIG. 1B, the opening 125 may also be where one or more remedies are applied from. In some embodiments, the opening 125 may allow for acne material from acne to be collected by the applicator, as shown in FIGS. 6A-6G.

In some embodiments, the first actuator 121 and the second actuator 122 perform different functions. For example, in some embodiments, the first actuator 121 may turn the applicator 100 on and/or off, while the second actuator 122 directs the applicator 100 to apply part of or all of the treatment. In some embodiments, the first actuator may direct the applicator 100 to apply a part of treatment (such as light treatment application), while the second actuator may direct the applicator 100 to apply a different part of treatment (such as suction of acne material). In some embodiments, the first actuator 121 and the second actuator 122 may be combined into a single actuator. In some embodiments, the first actuator 121, the second actuator 122, or both, may be a button, switch, touch-type capacitance button, or the like.

Button 123 may be configured to penetrate skin with one or more microneedles (such as shown in FIG. 1B). In some embodiments, by actuating the button, the microneedle is moved through the opening to penetrate the skin. In some embodiments, the button 123 is further configured to retain or remove the one or more microneedle. For example, in some embodiments, twisting the button 123 a first direction may retain the one or more microneedle, while twisting the button 123 in a second direction may release the one or more microneedle for easy, sanitary removal.

FIG. 1B is a cross section of the example applicator 100 of FIG. 1A, in accordance with the present technology. In some embodiments, the applicator 100 further includes one or more microneedles 105, a chamber 110, a reservoir 115, a valve 117, a formula tube 119, a processor 130, and a battery 133.

In some embodiments, the battery 133 is a rechargeable battery. In some embodiments, the battery 133 is configured to be electrically connected with a cable or wire to facilitate charging. In some embodiments, the battery 133 is an antenna configured to be charged wirelessly, such as through induction.

In some embodiments, the one or more microneedles 105 are configured to penetrate the skin. In some embodiments, the one or more microneedles 105 are configured to penetrate the skin at a depth of no more than 0.3 mm. In some embodiments, the one or more microneedles are configured to penetrate the skin by being pushed through the opening 125, such as with the button 123, as described in FIG. 1A. In some embodiments, the treatment plane 120 is shaped to allow a user to press the treatment plane 120 into the skin, and, due to skin's elasticity, penetrate the skin with the one or more microneedles 105. In other embodiments, the processor 130 may direct the one or more microneedles 105 to move through the opening 125 automatically. While the one or more microneedles 150 are illustrated as a cylinder in FIG. 1B, it should be understood that the applicator 100 may include any number of microneedles 105.

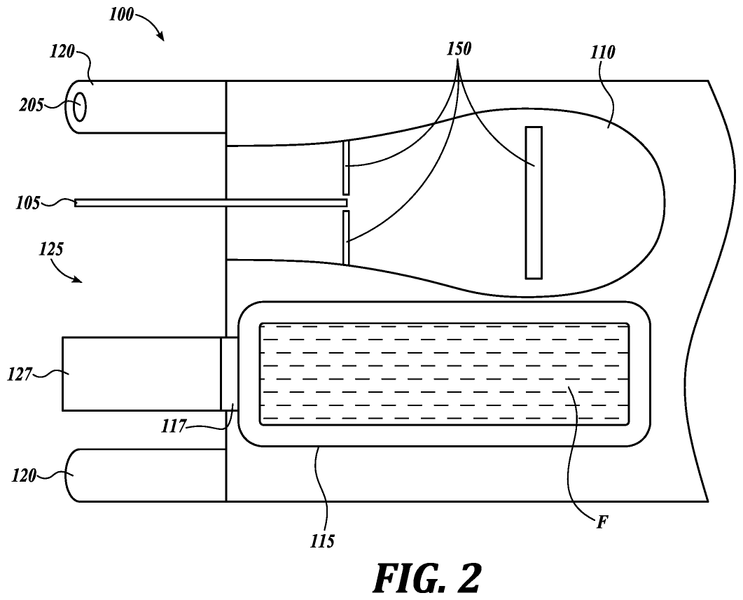

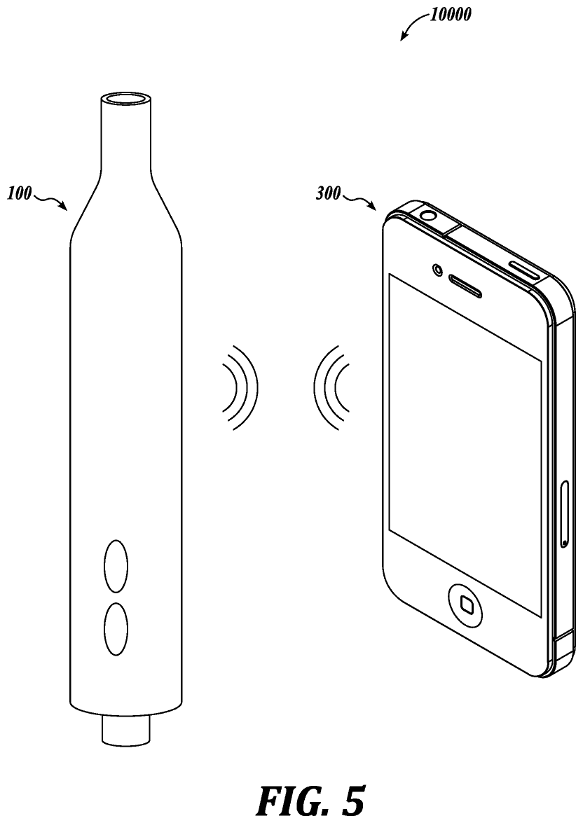

In some embodiments, the chamber 110 is configured to retain collected acne material. In some embodiments, the chamber 110 includes a suction, aspiration, or vacuum system, as shown and described in FIG. 2. In operation, the suction system pulls acne material from one or more acne into the chamber. In some embodiments, the chamber 110 is removable to allow for cleaning and/or sterilization. In some embodiments, the chamber 110 may detect an amount of acne material present in the chamber 110 and transmit a signal to the processor 130 and/or a smart device (such as shown in FIG. 5). In some embodiments, the processor 130 and/or the smart device may issue an alert that the chamber 110 is full and needs to be removed and cleaned and/or replaced.

In some embodiments, the applicator 100 includes a remedy system, including the reservoir 115, the valve 117, and the formula tube 119. In some embodiments, the remedy system may omit one or more of these components, or include additional components, as described and shown herein. In some embodiments, the reservoir 115 is configured to hold one or more formulas (or “remedies”). In some embodiments, the one or more formulas include hyaluronic acid, benzoyl peroxide, salicylic acid, retinoids, azelaic acid, antibiotics, or the like. In some embodiments, the one or more formulas are remedies for treating or preventing acne. In some embodiment, the one or more formulas are remedies for soothing irritation, such as cooling remedy or a heating remedy. In some embodiments, the valve 117 is fluidly connected to the reservoir 115 with a formula tube 119. In some embodiments, the formula tube 119 may be omitted, such as when the reservoir is shaped to contact the opening 125 and/or treatment plane 120, as shown in FIG. 2. In some embodiments, the one or more remedies may flow through the formula tube 119 to the valve 117, and then out the valve 117 either through the opening 125 or the treatment plane. In some embodiments, the reservoir 115 may be removed from applicator 100 to refill reservoir 115. In some embodiments, the reservoir 115 may be refilled while being retained in applicator 100.

In some embodiments, the processor 130 is communicatively coupled with one or more components of the applicator 100 (such as the remedy system, the suction system, the one or more microneedles 105, the first actuator 121, the second actuator 122, and/or the button 123). In some embodiments, the processor 130 is configured to apply treatment. In some embodiments, the processor 130 is further configured to issue alerts to the user. In some embodiments, the alerts may be tactile, visual, auditory, or the like. In some embodiments, the processor 130 is communicatively coupled with an external device, such as a smart device (as shown in FIG. 5).

FIG. 2 is a close-up cross-sectional view of an example applicator 100, in accordance with the present technology. In some embodiments, the applicator 100 includes one or more microneedles 105, a chamber 110, a reservoir 115, a valve 117, a treatment plane 120, an opening 125, a formula applicator 127, a suction system 150, and a light source 205.

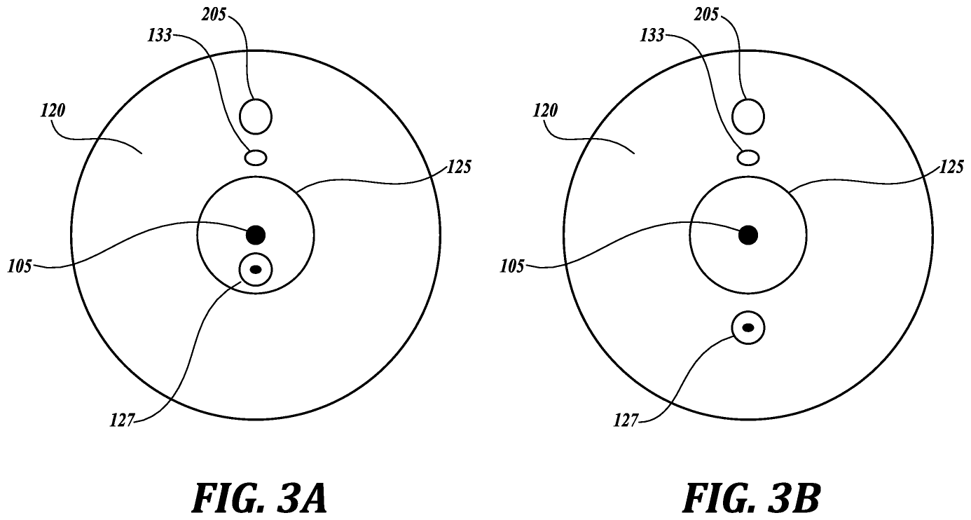

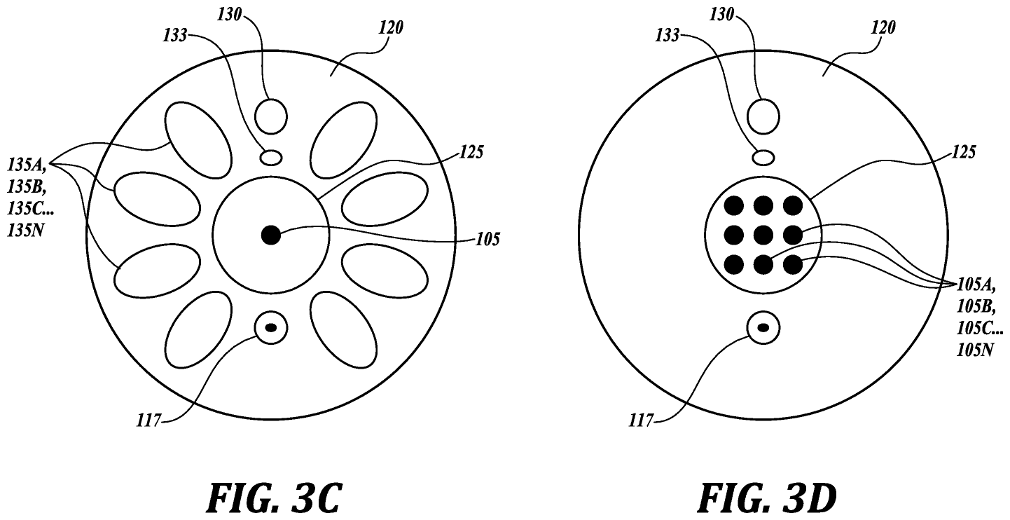

In some embodiments, the remedy system includes a reservoir 115, a valve 117, and a formula applicator 127. In some embodiments, one or more formulas F are extruded through the valve 117 and into the formula applicator 127. In some embodiments, the formula applicator 127 is configured to apply the one or more formulas F to the skin. In some embodiments, the formula applicator 127 is located inside the treatment plane 120, as shown in FIGS. 3A-3D. In some embodiments, the formula applicator 127 is at the same plane as the treatment plane 120 as shown in FIG. 2.

In some embodiments, the applicator 100 includes a suction system 150. In some embodiments, the suction system 150 may be a piezoelectric pump, such as shown in FIG. 2. In some embodiments, the suction system 150 may be any component capable of suctioning formula or material into the chamber 110. In some embodiments, the suction system 150 further includes a sensor configured to determine when to start and stop suctioning. In some embodiments, this is based on a predetermined time. In some embodiments, an optical sensor may be used to determine if acne material is still present. In some embodiments, preventing additional suctioning prevents damage to skin.

In some embodiments, the applicator 100 includes a light source 205. In some embodiments, the light source 205 is located on the treatment plane 120. In some embodiments, the light source 120 is a light emitting diode (LED). In some embodiments, the light source 205 is configured to emit a light treatment. In some embodiments, the light treatment is selected from blue light, near infrared (NIR) light, infrared (IR) light, ultraviolet (UV) light, or a combination thereof. In some embodiments, the light source 205 is configured to emit light having a peak emission in the visible light spectrum, the NIR spectrum, the UV spectrum, the IR spectrum, or a combination thereof. In some embodiments, the light source 170 has a peak emission wavelength from 170 to 400 nm, 570-590 nm, 630 to 700 nm, 800 to 1200 nm, or a combination thereof.

FIGS. 3A-3D are top-down perspectives of example applicator treatment planes 120, in accordance with the present technology. In some embodiments, the treatment plane 120 is circular, but in other embodiments, the treatment plane 120 may take any form, such as rectangular, square, or organically shaped. In some embodiments, the opening 125 is located in the center of the treatment plane 120. In some embodiments, the opening 125 may be off-center, such as close to an edge of the treatment plane 120. In some embodiments, the opening 125 is round, ovular, rectangular, square, or organically shaped.

In some embodiments, the applicator includes one or more microneedles 105, a formula applicator 127, an optical sensor 133, and a light source 205. In some embodiments, the optical sensor 133, the light source 205, and/or the formula applicator 127 may all be located on the treatment plane 120.

In some embodiments, the optical sensor 133 is a camera or a laser. In some embodiments, the optical sensor 133 is configured to collect image data (e.g., photos, videos, live feed, etc.) of the skin and transmit the image data to a processor (such as processor 130) and/or to an external device such as a smart device. The image data may be used to detect acne, direct treatment by the applicator, detect redness or irritation, or ensure that acne has been treated and/or reduced. In some embodiments, a smart device (such as shown in FIG. 5) may display image data in the form of images, video, and/or a live feed to assist a user in properly using the applicator.

FIG. 3A is an example applicator treatment plane 120, where the formula applicator 127 is located inside the opening 125 (as shown in detail in FIG. 2). In such embodiments, the formula applicator 127 is configured to apply one or more formulas (or “remedies”) to the skin. In such embodiments, the end of the formula applicator 127 may be on a same plane as the treatment plane, as shown in FIG. 2.

FIG. 3B is an example applicator treatment plane 120, where the formula applicator 127 is located on the treatment plane 120. In some embodiments, the formula applicator 127 is flush with the treatment plane. It may be advantageous to include the formula applicator 127 on the treatment plane 120 as opposed to inside the opening 125 to avoid contamination, mixing of the one or more remedies with acne material, and to allow for better suction by the suction system.

FIG. 3C is another example applicator treatment plane 120 including one or more massagers 135A, 135B, 135C . . . 135N. In some embodiments, the one or more massagers 135A, 135B, 135C . . . 135N may be located on the treatment plane 120. In some embodiments, the one or more massagers 135A, 135B, 135C . . . 135N are raised “bumps” or protrusions configured to contact the skin. In some embodiments, one or more massagers 135A, 135B, 135C . . . 135N may be used to massage the skin after treatment has been applied to reduce redness, swelling, discomfort, pain, or irritation. Massaging the skin after treatment has been applied may also further improve treatment by, for example, distributing the one or more remedies to the affected areas of the skin. In some embodiments, when the treatment plane 120 includes one or more massagers 135A, 135B, 135C . . . 135N, the one or more microneedle 105 is controlled with a button (such as button 123) and/or the processor 130, so that the microneedle 105 extends past the treatment plane 120, so that the one or more massagers 135A, 135B, 135C . . . 135N do not contact the skin when the one or more microneedle 105 penetrates the skin. While the one or more massagers 135A, 135B, 135C . . . 135N are illustrated as ovular, it should be understood that the one or more massagers 135A, 135B, 135C . . . 135N may take any form, such as dome-shaped, prismatic, organically shaped, including one or more “fingers”, or the like. In some embodiments, the one or more microneedles 105 are covered with a cap or cover, to prevent the one or more microneedles 105 from being damaged.

FIG. 3D is an example applicator treatment plane 120, where the applicator includes a plurality of microneedles 105A, 105B, 105C . . . 105N. In some embodiments, the plurality of microneedles 105A, 105B, 105C . . . 105N is arranged in an array in the opening 125. In some embodiments, the plurality of microneedles 105A, 105B, 105C . . . 105N is otherwise arranged, such as in a ring, a diamond shaped array, a circular array, or the like. In some embodiments, the one or more microneedles 105 are coupled with the processor 130. In such embodiments, the processor 130 is configured to determine if the needle is dirty, damaged, or wet. In some embodiments, the processor 130 issues an alert when wetness or damage is detected. In some embodiments, wetness or damage is detected with capacitance of the one or more microneedles 105.

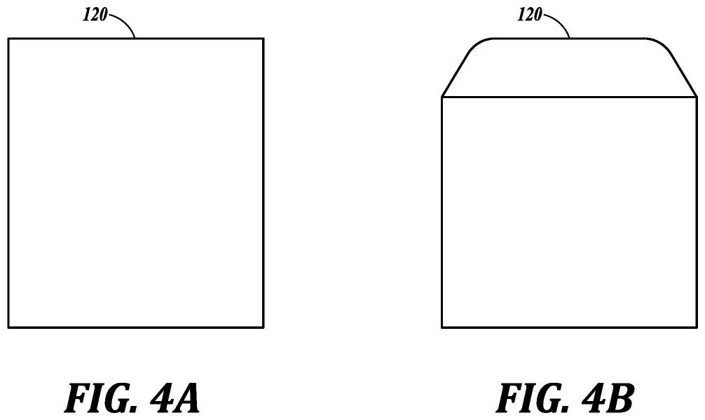

FIGS. 4A-4B are side-view perspectives of example applicator treatment planes 120, in accordance with the present technology. In some embodiments, the treatment plane 120 is flat, as shown in FIG. 4A. In such embodiments, the flat of the treatment plane 120 contacts the skin. In other embodiments, the treatment plane 120 is rounded, such as shown in FIG. 4B. In such embodiments, the rounded area of the treatment plane 120 may act as a massager.

FIG. 5 is an example system 1000 for treating acne, in accordance with the present technology. In some embodiments, the system 1000 includes an applicator 100 as described and shown in FIGS. 1A-4B. In some embodiments, the system 1000 further includes a smart device 300.

In some embodiments, the applicator 100 and the smart device 300 are communicatively coupled. In some embodiments, the applicator 100 and the smart device 300 are coupled through a wireless network, such as Wifi or Bluetooth™. In some embodiments, the applicator 100 and the smart device 300 may be otherwise communicatively coupled, such as with a cable or wire.

In some embodiments, the smart device 300 is a smartphone, computer, laptop, tablet, or the like. In some embodiments, the smart device 300 includes an application. In some embodiments, the application includes a treatment plan, a record of each instance of treatment, a recommendation for applying or not applying the microneedle treatment, and the like, as explained in detail herein.

In some embodiments, the smart device 300 is configured to store treatment data of the applicator, where the treatment data comprises an instance of treatment. In some embodiments, the smart device is further configured to detect one or more indicators of irritation on the skin, and, when the one or more indicators of irritation are detected, prevent treatment. In this manner, the smart device 300 can prevent a user from overusing the applicator 100 and causing further irritation or damage to their skin.

In some embodiments, the smart device 300 is further configured to determine an amount of time between a first treatment and a second treatment, compare the amount of time to a time threshold, and, when the amount of time is below the time threshold, prevent treatment. In this manner, the smart device can prevent a user from overusing the applicator 100.

In some embodiments, the smart device 300 can determine if the user of the applicator 100 is a same user of the applicator, for example, through a password, fingerprint, or other bioinformatic. In some embodiments, if a different user is determined to be using the applicator 100, the smart device 300 can prevent the different user from applying treatment. In this manner, the applicator 100 can prevent contamination, and ensure sanitary use of the applicator 100. In some embodiments, the applicator 100 may include multiple authorized users. In such embodiments, the applicator 100 may require authorization or identification of each user. In some embodiments, the applicator 100 received authorization or identification for a user and either initiates treatment or prevents treatment until the applicator 100 is cleaned. In some embodiments, the smart device 300 or the applicator 100 directs the user to clean the applicator 100.

FIGS. 6A-6G are process diagrams of the applicator 100 of FIG. 2 in use, in accordance with the present technology. In some embodiments, the applicator 100 includes a treatment plane 120, a light source 203, one or more microneedle 105, a chamber 110, a suction system 150, a reservoir 115, a valve 117, and a formula applicator 127. Applicator 100 is only an example of the components that may be included or omitted from the applicator 100. It should be understood that any applicator described and/or shown herein may perform the process illustrated in FIG. 6A-6G.

FIG. 6A the applicator 100 (such as through an optical sensor as shown in FIGS. 3A-3D) detects acne A on skin S. In some embodiments, the applicator 100 and/or a smart device (such as smart device 300) issues an alert 10010 that acne A has been detected. In some embodiments, alert 10010 is a tactile alert, such as vibration, an auditory alert, such as a chime or time, a visual alert, such as a light flash or a pop up on the smart device, or a combination thereof. In some embodiments, the smart device may direct a user to the acne A, and/or tell the user to apply treatment. In some embodiments, the applicator 100 may automatically begin treatment when the acne A is detected. In some embodiments, the area of skin where acne A is present is considered an “affected area”.

In FIG. 6B, the applicator 100 is placed on the skin S. The treatment plane 120 contacts the skin, and the one or more microneedle 105 penetrates the skin S at the acne A (or “affected area”). As explained herein, in some embodiments, the one or more microneedle 105 penetrates the skin after being directed to do so by the processor (such as processor 103) and/or smart device. In some embodiments, the one or more microneedle penetrates the skin S penetrates the skin S when a button (such as button 123) is pressed. In yet other embodiments, the one or more microneedle 105 penetrates the skin when a user presses the treatment plane 120 firmly against the skin S. In some embodiments, a combination of these mechanisms may be implemented.

In FIG. 6C, the suction system 150 removes the acne material AM from the skin S, and suctions the acne material AM into the chamber 110. When the acne material AM is suctioned away from the acne A, a treated zone TZ is left on/in the skin. In some embodiments, the suction system forms a partial vacuum. In some embodiments, the suction system is a pump, such as an electrothermal pump, a solenoid pump, an electric pump, or a piezoelectric pump.

In FIG. 6D, the applicator 100 is removed from the skin S as the acne material AM is retained in the chamber.

In FIG. 6E, light treatment LT is applied to the treated zone TZ with the light source 205. In some embodiments, the light treatment LT is blue light, red light, near infrared (NIR) light, infrared (IR) light, ultraviolet (UV) light or a combination thereof. In some embodiments, the applicator 100 and/or the smart device direct the user one how to move the applicator 100 to apply the light treatment to the treated zone TZ (or “affected area”).

In FIG. 6F, one or more remedies (or “formulas”) F is applied from the reservoir 115 through a valve 117 and through the formula applicator 127 onto the skin S. In some embodiments, the process diagrams of FIG. 6E and FIG. 6F may be switched, i.e., so that the one or more remedies are applied to the treated zone TZ before the light treatment TZ.

In FIG. 6G, the applicator 100 is moved about the skin S, as shown by the arrows, so that the treatment plane 120 (and one or more massagers located on the treatment plane, such as shown in FIG. 3C) contact the skin. In some embodiments, the applicator 100 is moved across both the treatment zone TZ and the surrounding skin S. In some embodiments, massaging the skin spreads the one or more remedies F onto the skin S. In some embodiments, massaging the skin reduces irritation or pain.

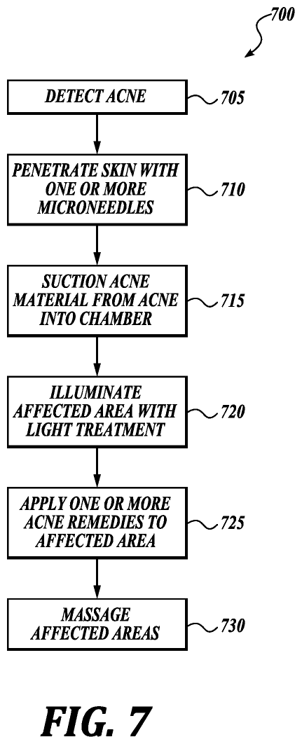

FIG. 7 is an example method 700 of using the applicator and/or system described herein, in accordance with the present technology. In some embodiments, the method 700 is carried out with an applicator (such as applicator 100) and/or a smart device (such as smart device 300).

In some embodiments, the applicator includes an optical sensor (such as optical sensor 133), one or more microneedles (such as one or more microneedles 105 or plurality of microneedles 105A, 105B, 105C . . . 105N), a treatment plane (such as treatment plane 120), a suction system (such as suction system 150), a chamber (such as chamber 110), a reservoir (such as reservoir 115), and a valve (such as valve 117). In some embodiments, the applicator may further include a formula tube (such as formula tube 119) and/or a formula applicator (such as formula applicator 127). In some embodiments, the applicator includes one or more massagers (such as one or more massager 135A, 135B, 135C . . . 135N).

In block 705, acne (such as acne A) is detected. In some embodiments, the acne is detected with the optical sensor of the applicator. In some embodiments, the optical sensor collects image data of skin (such as skin S) and a processor (such as processor 130) of the applicator and/or the smart device determine if acne is present on the skin. In some embodiments, the applicator and/or the smart device issues an alert as described herein that acne has been detected.

In block 710, the skin is penetrated with one or more microneedles. As described herein, the one or more microneedles may penetrate the skin through pressure, through the pressing of a button (such as button 123) on the applicator, and/or through direction by the processer and/or smart device.

In block 715, acne material is suctioned into the chamber of the applicator. In some embodiments, this is done with the suction system. The suction system may include a pump, such as an electrochemical pump, an electric pump, a solenoid pump, or a piezoelectric pump.

In block 720, the affected area (i.e., the acne, treated zone (such as treated zone TZ) and/or the surrounding skin) is illuminated with light treatment. In some embodiments, the light treatment is blue light, NIR light, IR light, UV light, or a combination thereof.

In block 725, one or more acne remedies (such as formula F) are applied to the affected area. In some embodiments, the one or more acne remedies include hyaluronic acid, benzoyl peroxide, salicylic acid, retinoids, azelaic acid, antibiotics, or the like. In some embodiments, the one or more remedies are configured to treat or prevent acne. In some embodiment, the one or more formulas are remedies for soothing irritation, such as cooling remedy or a heating remedy.

In block 730, optionally, the skin is massaged with the treatment plane and/or one or more massagers located on the treatment plane. In some embodiments, massaging the skin further spreads the one or more remedies onto the skin. It should be understood that block 720, 725, and 730 may be performed in any order.

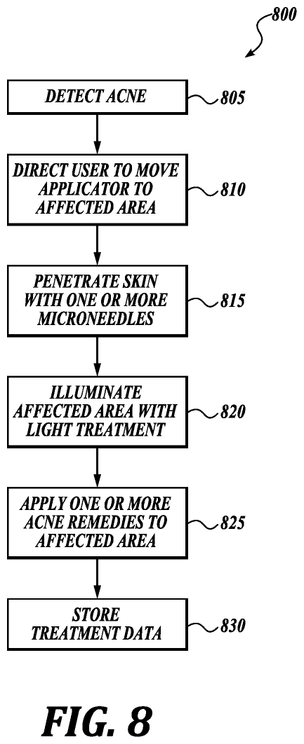

FIG. 8 is an example method 800 of using the applicator and/or system described herein, in accordance with the present technology. In some embodiments, the method 800 is carried out with an applicator (such as applicator 100) and/or a smart device (such as smart device 300).

In some embodiments, the applicator includes an optical sensor (such as optical sensor 133), one or more microneedles (such as one or more microneedles 105 or plurality of microneedles 105A, 105B, 105C . . . 105N), a treatment plane (such as treatment plane 120), a suction system (such as suction system 150), a chamber (such as chamber 110), a reservoir (such as reservoir 115), and a valve (such as valve 117). In some embodiments, the applicator may further include a formula tube (such as formula tube 119) and/or a formula applicator (such as formula applicator 127). In some embodiments, the applicator includes one or more massagers (such as one or more massager 135A, 135B, 135C . . . 135N).

In block 805, acne (such as acne A) is detected. In some embodiments, the acne is detected with the optical sensor of the applicator. In some embodiments, the optical sensor collects image data of skin (such as skin S) and a processor (such as processor 130) of the applicator and/or the smart device determine if acne is present on the skin. In some embodiments, the applicator and/or the smart device issues an alert as described herein that acne has been detected.

In block 810, the user is directed to move the applicator to the affected area (i.e., the acne, treated zone (such as treated zone TZ) and/or the surrounding skin). In some embodiments, the user is directed with an alert (such as alert 10100) by the applicator and/or the smart device. In some embodiments, the smart device may display or guide a user to the affected area, such as with a live feed from the optical sensor.

In block 815, the skin is penetrated with one or more microneedles. As described herein, the one or more microneedles may penetrate the skin through pressure, through the pressing of a button (such as button 123) on the applicator, and/or through direction by the processer and/or smart device.

In block 820, the affected area is illuminated with light treatment. In some embodiments, the light treatment is blue light, NIR light, IR light, UV light, or a combination thereof.

In block 825, one or more acne remedies (such as formula F) are applied to the affected area. In some embodiments, the one or more acne remedies include hyaluronic acid, benzoyl peroxide, salicylic acid, retinoids, azelaic acid, antibiotics, or the like. In some embodiments, the one or more remedies are configured to treat or prevent acne. In some embodiment, the one or more formulas are remedies for soothing irritation, such as cooling remedy or a heating remedy. It should be understood that blocks 820 and 825 may be performed in any order. Further in some embodiments, the method may include suctioning acne material (as explained in block 715 of FIG. 7) and/or massaging the affected areas (as explained in block 730 of FIG. 7).

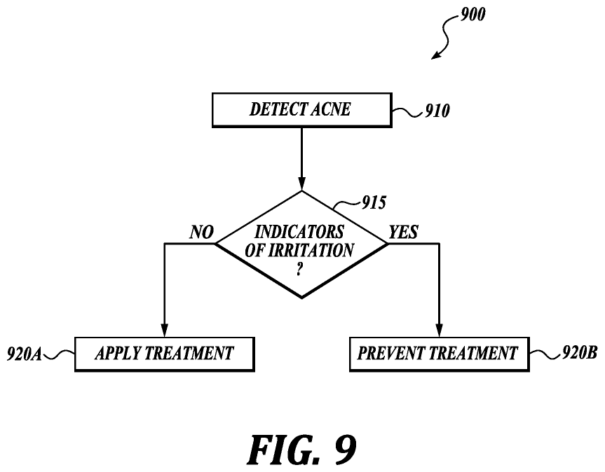

In block 830, treatment data is stored. In some embodiments, the treatment data is each instance of treatment applied by the applicator. As described herein, treatment includes penetrating the skin with one or more microneedles, suctioning acne material with a suction mechanism, applying light treatment to one or more affected areas, and applying one or more remedies (or “formulas”) to the affected areas. In some embodiments, each instance of treatment is each instance of the microneedle penetrating the skin. In some embodiments, each instance of treatment is each acne or affected area treated. In some embodiments, each instance of treatment is the time between when a user begins treatments to the skin and when the user ends treatments to the skin. In some embodiments, the treatment data is stored by the processor of the applicator and/or by the smart device. In some embodiments, the stored treatment data may be used to suggest, modify, or even prevent use with the applicator, as explained in FIGS. 9-11.

FIG. 9 is an example treatment method 900, in accordance with the present technology. In some embodiments, the method 900 is carried out with an applicator (such as applicator 100) and/or a smart device (such as smart device 300).

In some embodiments, the applicator includes an optical sensor (such as optical sensor 133), one or more microneedles (such as one or more microneedles 105 or plurality of microneedles 105A, 105B, 105C . . . 105N), a treatment plane (such as treatment plane 120), a suction system (such as suction system 150), a chamber (such as chamber 110), a reservoir (such as reservoir 115), and a valve (such as valve 117). In some embodiments, the applicator may further include a formula tube (such as formula tube 119) and/or a formula applicator (such as formula applicator 127). In some embodiments, the applicator includes one or more massagers (such as one or more massager 135A, 135B, 135C . . . 135N).

It should be understood that in some embodiments, the method 900 occurs after the methods 700 and/or 800. Further, method 900 can be carried out either simultaneously or sequentially with methods 1000 and 1100.

In block 905, acne (such as acne A) is detected. In some embodiments, the acne is detected with the optical sensor of the applicator. In some embodiments, the optical sensor collects image data of skin (such as skin S) and a processor (such as processor 130) of the applicator and/or the smart device determine if acne is present on the skin. In some embodiments, the applicator and/or the smart device issues an alert as described herein that acne has been detected.

In decision block 915, it is determined whether indicators of irritation are present on the skin. In some embodiments, indicators of irritation include redness (separate from that associated with acne), wounds, bleeding, or the like. In some embodiments, the indicators of irritation are detected by the optical sensor. In some embodiments, the applicator may include additional sensors such as temperature sensors for further determining if irritation is indicated on the skin. If indicators of irritation are not present, the method proceeds to block 920A.

In block 920A, treatment is applied. Treatment includes penetrating the skin with one or more microneedles, suctioning acne material with a suction mechanism, applying light treatment to one or more affected areas, and/or applying one or more remedies (or “formulas”) to the affected areas.

Returning to decision block 915, if indicators of irritation are detected, the method proceeds to block 920B. In some embodiments, there is an irritation threshold. In some embodiments, the processor and/or smart device may compare the detected indicators of irritation to the irritation threshold, and only proceed to block 920B if the irritation meets or exceeds the irritation threshold. Example thresholds for irritation include a temperature threshold and/or a redness threshold, such as darker redness associated with bleeding.

In block 920B, treatment is prevented. In some embodiments, the processor and/or smart device turns the applicator off. In some embodiments, treatment is prevented by retracting or retaining the one or more microneedle. In this manner, the applicator and/or smart device can prevent further irritation. In some embodiments, only a portion of the treatment is prevented. For example, in some embodiments, only the microneedle treatment is prevented. In such embodiments, the applicator still allows the dispensing of remedies and/or light treatment.

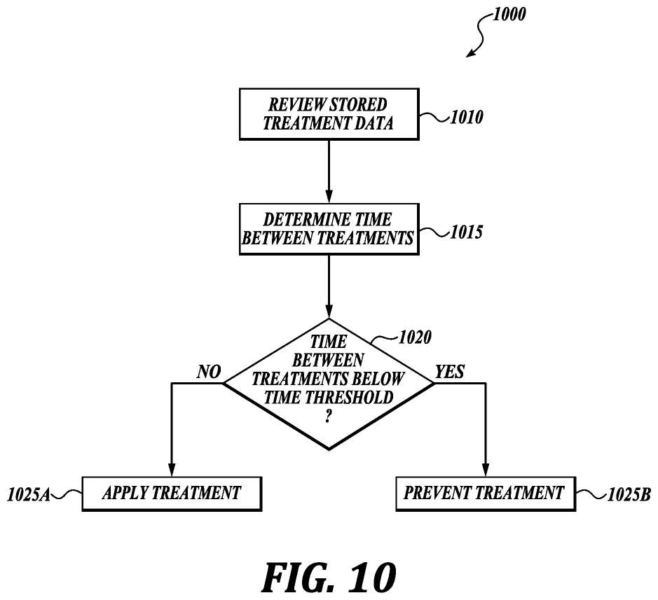

FIG. 10 is another example treatment method 1000, in accordance with the present technology. In some embodiments, the method 1000 is carried out with an applicator (such as applicator 100) and/or a smart device (such as smart device 300).

In some embodiments, the applicator includes an optical sensor (such as optical sensor 133), one or more microneedles (such as one or more microneedles 105 or plurality of microneedles 105A, 105B, 105C . . . 105N), a treatment plane (such as treatment plane 120), a suction system (such as suction system 150), a chamber (such as chamber 110), a reservoir (such as reservoir 115), and a valve (such as valve 117). In some embodiments, the applicator may further include a formula tube (such as formula tube 119) and/or a formula applicator (such as formula applicator 127). In some embodiments, the applicator includes one or more massagers (such as one or more massager 135A, 135B, 135C . . . 135N).

It should be understood that in some embodiments, the method 1000 occurs after the methods 700 and/or 800. Further, method 1000 can be carried out either simultaneously or sequentially with methods 900 and 1100.

In block 1010, as the user turns on the applicator, the processor and/or the smart device reviews stored treatment data. In some embodiments, each instance of treatment is each instance of the microneedle penetrating the skin. In some embodiments, each instance of treatment is each acne or affected area treated. In some embodiments, each instance of treatment is the time between when a user begins treatments to the skin and when the user ends treatments to the skin. In some embodiments, the treatment data is stored by the processor of the applicator and/or by the smart device.

In block 1015, a time between treatments is determined. In some embodiments, the applicator and/or the smart device includes a time threshold, where treatment cannot be applied if there has not been enough time between instances of treatment.

In decision block 1020, the time between instances of treatment is compared to the time threshold, and it is determined whether the time between instances of treatment is below the time threshold. In some embodiments, the time between instances of treatment is the time between the treatment of the same acne. For example, in some embodiments, the applicator and/or smart device stores data for each time an individual acne (such as a pimple or blackhead) is treated and prevents treatment of that same individual acne until the time threshold has passed. In such embodiments, the time threshold may be, for example, 6-12 hours. In some embodiments, such as when treatment is the time between when a user begins treatments to the skin and when the user ends treatments to the skin, the time threshold may prevent continued treatment to a user's skin. In such embodiments, the time threshold may be, for example, 6-72 hours. If the time is not below the time threshold, the method proceeds to block 1025A.

In block 1025A, treatment is applied. Treatment includes penetrating the skin with one or more microneedles, suctioning acne material with a suction mechanism, applying light treatment to one or more affected areas, and/or applying one or more remedies (or “formulas”) to the affected areas.

Returning to decision block 1020, if the time between instances of treatment is below the time threshold, the method proceeds to block 1025B.

In block 1025B, treatment is prevented. In some embodiments, the processor and/or smart device turns the applicator off. In some embodiments, treatment is prevented by retracting or retaining the one or more microneedle. In this manner, the applicator and/or smart device can prevent irritation, injury, and/or overuse of the applicator. In some embodiments, only a portion of the treatment is prevented. For example, in some embodiments, only the microneedle treatment is prevented. In such embodiments, the applicator still allows the dispensing of remedies and/or light treatment.

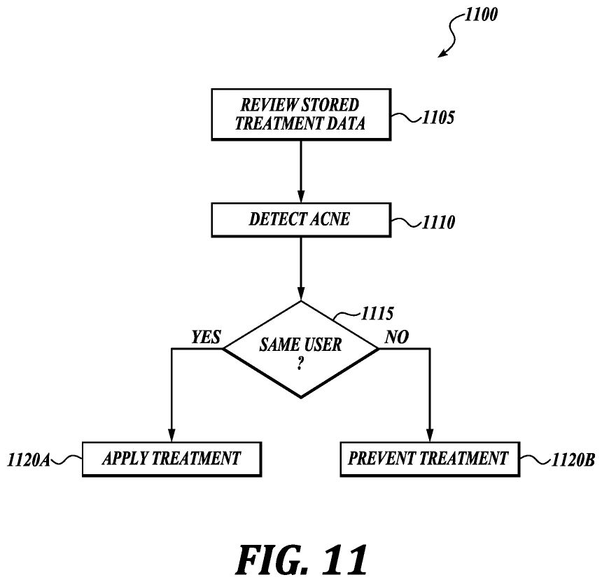

FIG. 11 is yet another example treatment method 1100, in accordance with the present technology. In some embodiments, the method 1100 is carried out with an applicator (such as applicator 100) and/or a smart device (such as smart device 300).

In some embodiments, the applicator includes an optical sensor (such as optical sensor 133), one or more microneedles (such as one or more microneedles 105 or plurality of microneedles 105A, 105B, 105C . . . 105N), a treatment plane (such as treatment plane 120), a suction system (such as suction system 150), a chamber (such as chamber 110), a reservoir (such as reservoir 115), and a valve (such as valve 117). In some embodiments, the applicator may further include a formula tube (such as formula tube 119) and/or a formula applicator (such as formula applicator 127). In some embodiments, the applicator includes one or more massagers (such as one or more massager 135A, 135B, 135C . . . 135N).

It should be understood that in some embodiments, the method 1100 occurs after the methods 700 and/or 800. Further, method 1100 can be carried out either simultaneously or sequentially with methods 900 and 1000.

In block 1105, as the user turns on the applicator, the processor and/or the smart device reviews stored treatment data. In some embodiments, each instance of treatment is each instance of the microneedle penetrating the skin. In some embodiments, each instance of treatment is each acne or affected area treated. In some embodiments, each instance of treatment is the time between when a user begins treatments to the skin and when the user ends treatments to the skin. In some embodiments, the treatment data is stored by the processor of the applicator and/or by the smart device.

In block 1110, acne (such as acne A) is detected. In some embodiments, the acne is detected with the optical sensor of the applicator. In some embodiments, the optical sensor collects image data of skin (such as skin S) and a processor (such as processor 130) of the applicator and/or the smart device determine if acne is present on the skin. In some embodiments, the applicator and/or the smart device issues an alert as described herein that acne has been detected.

In decision block 1115, it is determined whether a same user is attempting to apply the treatment. In some embodiments, the applicator and/or the smart device can determine an identity of a user of the device. In some embodiments, this is with a fingerprint, a password, an account, or the like. In some embodiments, the applicator and/or the smart device can request an identity of the user through requesting an authorization such as the password, account information, or fingerprint before allowing treatment. In some embodiments, the applicator may include multiple authorized users. In such embodiments, the applicator may require authorization or identification of each user. In some embodiments, the applicator received authorization or identification for a user and either initiates treatment or prevents treatment until the applicator is cleaned. In some embodiments, the smart device or the applicator directs the user to clean the applicator. In some embodiments, the applicator and/or smart device can compare past optical data of previous instances of treatment with image data received by the applicator at the time of the desired treatment. If the applicator and/or the smart device determines that the user is the same user, the method proceeds to block 1120A.

In block 1120A, treatment is applied. Treatment includes penetrating the skin with one or more microneedles, suctioning acne material with a suction mechanism, applying light treatment to one or more affected areas, and/or applying one or more remedies (or “formulas”) to the affected areas.

Returning to decision block 1115, if the user is determined to be a different user, the method proceeds to block 1120B.

In block 1120B, treatment is prevented. In some embodiments, the processor and/or smart device turns the applicator off. In some embodiments, treatment is prevented by retracting or retaining the one or more microneedle. In this manner, the applicator and/or smart device can prevent contamination of the applicator. In some embodiments, only a portion of the treatment is prevented. For example, in some embodiments, only the microneedle treatment is prevented. In such embodiments, the applicator still allows the dispensing of remedies and/or light treatment.

It should be understood that all methods 700, 800, 900, 1000, and 1100 should be interpreted as merely representative. In some embodiments, process blocks of all methods 700, 800, 900, 1000, and 1100 may be performed simultaneously, sequentially, in a different order, or even omitted, without departing from the scope of this disclosure.

The present application may reference quantities and numbers. Unless specifically stated, such quantities and numbers are not to be considered restrictive, but representative of the possible quantities or numbers associated with the present application. Also, in this regard, the present application may use the term “plurality” to reference a quantity or number. In this regard, the term “plurality” is meant to be any number that is more than one, for example, two, three, four, five, etc. The terms “about,” “approximately,” “near,” etc., mean plus or minus 5% of the stated value. For the purposes of the present disclosure, the phrase “at least one of A, B, and C,” for example, means (A), (B), (C), (A and B), (A and C), (B and C), or (A, B, and C), including all further possible permutations when greater than three elements are listed.

Embodiments disclosed herein may utilize circuitry in order to implement technologies and methodologies described herein, operatively connect two or more components, generate information, determine operation conditions, control an appliance, device, or method, and/or the like. Circuitry of any type can be used. In an embodiment, circuitry includes, among other things, one or more computing devices such as a processor (e.g., a microprocessor), a central processing unit (CPU), a digital signal processor (DSP), an application-specific integrated circuit (ASIC), a field-programmable gate array (FPGA), or the like, or any combinations thereof, and can include discrete digital or analog circuit elements or electronics, or combinations thereof.

An embodiment includes one or more data stores that, for example, store instructions or data. Non-limiting examples of one or more data stores include volatile memory (e.g., Random Access memory (RAM), Dynamic Random Access memory (DRAM), or the like), non-volatile memory (e.g., Read-Only memory (ROM), Electrically Erasable Programmable Read-Only memory (EEPROM), Compact Disc Read-Only memory (CD-ROM), or the like), persistent memory, or the like. Further non-limiting examples of one or more data stores include Erasable Programmable Read-Only memory (EPROM), flash memory, or the like. The one or more data stores can be connected to, for example, one or more computing devices by one or more instructions, data, or power buses.

In an embodiment, circuitry includes a computer-readable media drive or memory slot configured to accept signal-bearing medium (e.g., computer-readable memory media, computer-readable recording media, or the like). In an embodiment, a program for causing a system to execute any of the disclosed methods can be stored on, for example, a computer-readable recording medium (CRMM), a signal-bearing medium, or the like. Non-limiting examples of signal-bearing media include a recordable type medium such as any form of flash memory, magnetic tape, floppy disk, a hard disk drive, a Compact Disc (CD), a Digital Video Disk (DVD), Blu-Ray Disc, a digital tape, a computer memory, or the like, as well as transmission type medium such as a digital and/or an analog communication medium (e.g., a fiber optic cable, a waveguide, a wired communications link, a wireless communication link (e.g., transmitter, receiver, transceiver, transmission logic, reception logic, etc.). Further non-limiting examples of signal-bearing media include, but are not limited to, DVD-ROM, DVD-RAM, DVD+RW, DVD-RW, DVD-R, DVD+R, CD-ROM, Super Audio CD, CD-R, CD+R, CD+RW, CD-RW, Video Compact Discs, Super Video Discs, flash memory, magnetic tape, magneto-optic disk, MINIDISC, non-volatile memory card, EEPROM, optical disk, optical storage, RAM, ROM, system memory, web server, or the like.

The detailed description set forth above in connection with the appended drawings, where like numerals reference like elements, are intended as a description of various embodiments of the present disclosure and are not intended to represent the only embodiments. Each embodiment described in this disclosure is provided merely as an example or illustration and should not be construed as preferred or advantageous over other embodiments. The illustrative examples provided herein are not intended to be exhaustive or to limit the disclosure to the precise forms disclosed. Similarly, any steps described herein may be interchangeable with other steps, or combinations of steps, in order to achieve the same or substantially similar result. Generally, the embodiments disclosed herein are non-limiting, and the inventors contemplate that other embodiments within the scope of this disclosure may include structures and functionalities from more than one specific embodiment shown in the figures and described in the specification.

In the foregoing description, specific details are set forth to provide a thorough understanding of exemplary embodiments of the present disclosure. It will be apparent to one skilled in the art, however, that the embodiments disclosed herein may be practiced without embodying all the specific details. In some instances, well-known process steps have not been described in detail in order not to unnecessarily obscure various aspects of the present disclosure. Further, it will be appreciated that embodiments of the present disclosure may employ any combination of features described herein.

The present application may include references to directions, such as “vertical,” “horizontal,” “front,” “rear,” “left,” “right,” “top,” and “bottom,” etc. These references, and other similar references in the present application, are intended to assist in helping describe and understand the particular embodiment (such as when the embodiment is positioned for use) and are not intended to limit the present disclosure to these directions or locations.

The present application may also reference quantities and numbers. Unless specifically stated, such quantities and numbers are not to be considered restrictive, but exemplary of the possible quantities or numbers associated with the present application. Also, in this regard, the present application may use the term “plurality” to reference a quantity or number. In this regard, the term “plurality” is meant to be any number that is more than one, for example, two, three, four, five, etc. The term “about,” “approximately,” etc., means plus or minus 5% of the stated value. The term “based upon” means “based at least partially upon.”

The principles, representative embodiments, and modes of operation of the present disclosure have been described in the foregoing description. However, aspects of the present disclosure, which are intended to be protected, are not to be construed as limited to the particular embodiments disclosed. Further, the embodiments described herein are to be regarded as illustrative rather than restrictive. It will be appreciated that variations and changes may be made by others, and equivalents employed, without departing from the spirit of the present disclosure. Accordingly, it is expressly intended that all such variations, changes, and equivalents fall within the spirit and scope of the present disclosure as claimed.

While illustrative embodiments have been illustrated and described, it will be appreciated that various changes can be made therein without departing from the spirit and scope of the invention.

1. An applicator, comprising:
one or more microneedles;
an optical sensor configured to detect acne;
a removal system comprising:
a suction mechanism configured to remove acne material, and
a chamber for containing the acne material;
a remedy system configured apply one or more acne remedies;
a light source configured to apply a light stimulus to skin; and
a processor configured to:
detect acne at one or more affected areas on skin; and
direct the applicator to apply treatment, wherein the treatment comprises:
penetrating the skin at the one or more affected areas with the one or more microneedles;
removing acne material with the suction mechanism;
applying the light stimulus to the one or more affected areas; and
applying one or more acne remedies to the one or more affected areas.

2. The applicator of claim 1, further comprising a treatment plane configured to contact the skin, wherein the treatment plane surrounds the one or more microneedles.

3. The applicator of claim 2, further comprising one or more massagers located on the treatment plane, wherein the one or more massagers are configured to massage the skin.

4. The applicator of claim 2, wherein the treatment plane is rounded.

5. The applicator of claim 2, wherein the light source is disposed on the treatment plane.

6. The applicator of claim 1, wherein the suction mechanism comprises a piezoelectric pump.

7. The applicator of claim 1, wherein the remedy system comprises:
one or more reservoirs configured to hold the one or more remedies; and
a valve configured to apply the one or more remedies to skin.

8. The applicator of claim 6, wherein the valve is disposed on the treatment plane.

9. The applicator of claim 1, wherein the one or more microneedles are a plurality of microneedles arranged in an array.

10. The applicator of claim 1, wherein the light source is configured to emit blue light, near infrared (NIR) light, infrared (IR) light, ultraviolet (UV) light, or a combination thereof.

11. A system for treating acne, comprising:
the applicator of claim 1; and
a smart device communicatively coupled to the applicator.

12. The system of claim 11, wherein the smart device is configured to store treatment data of the applicator, wherein the treatment data comprises an instance of treatment.

13. The system of claim 11, wherein the smart device is further configured to:
detect one or more indicators of irritation on the skin; and
when the one or more indicators of irritation are detected, prevent treatment.

14. The system of claim 11, wherein the smart device is further configured to:
determine an amount of time between a first treatment and a second treatment;
compare the amount of time to a time threshold; and
when the amount of time is below the time threshold, prevent treatment.

15. A method of treating acne, the method comprising:
detecting acne at one or more affected areas;
applying treatment, wherein treatment comprises:
penetrating skin with one or more microneedles;
suctioning acne material from the acne;
illuminating the one or more affected areas with light treatment; and
applying one or more acne remedies to the one or more affected areas.

16. The method of claim 15, wherein the method further comprises massaging the one or more affected areas.

17. The method of claim 15, wherein the method further comprises storing treatment data, wherein the treatment data comprises an instance of the treatment.

18. The method of claim 15, wherein the method further comprises:
detecting one or more indicators of irritation on the skin; and,
when the one or more indicators of irritation are detected, preventing treatment.

19. The method of claim 15, wherein the method further comprises:
determining an amount of time between a first treatment and a second treatment;
comparing the amount of time to a time threshold; and,
when the amount of time is below the time threshold. preventing treatment.

20. The method of claim 15, wherein the method further comprises:
determining if a user of the applicator is a same user associated with the stored treatment; and,
when the user is not the same user, preventing treatment.
